(19) United States
(12) Patent Application Publication
(10) Pub. No.: US 2006/0058217 A1
(43) Pub. Date: Mar. 16, 2006

(54) Compositions and methods for treatment of ovarian cancer

(76) Inventors: Hillary D White, S. Pomfret, VT (US); Katherine Fern Terranova, Overland Park, KS (US)

Correspondence Address: LICATLA & TYRRELL P.C., 66 E. MAIN STREET, MARLTON, NJ, 08053, US

(21) Appl. No.: 10/541,474

(22) PCT Filed: Jan. 20, 2004
(86) PCT No.: PCT/US04/01382

§ 371 Date: Sep. 9, 2005

Related U.S. Application Data
(60) Provisional application No. 60/441,616, filed on Jan. 21, 2003.

(51) Int. Cl. A61K 38/17

(52) U.S. Cl. 514/2

(57) ABSTRACT

Compositions and methods for treatment of ovarian cancer are described. The compositions comprise antalamin, a compound that increases cytotoxic T-cell lytic activity at tumor sites. Methods of treatment for ovarian cancer are based on the administration of antalarmin to increase cytotoxic T-cell lytic activity or anti-tumor activity, which leads to killing of tumor cells by cytotoxic T cells.

### BACKGROUND OF THE INVENTION

Ovarian carcinoma is one of the most lethal gynecologic cancers and is considered to be the most problematic female cancer in terms of diagnosis and treatment. Approximately 26,000 women in the United States are diagnosed every year with ovarian carcinoma and only half will survive five years. There remains a need for effective treatments for ovarian cancer.

Ninety percent of human ovarian cancers are derived from ovarian surface epithelium. The ovarian surface epithelium is derived from the embryonic celomic epithelium of the gonadal ridge and adjacent areas, which then differentiates into granulosa cells, oviductal, endometrial and endocervical epithelia, and ovarian surface epithelium. The ovarian surface epithelium is complex, with involvement in post-ovulatory repair and restoration of the ovary. The surface epithelial cells are pluripotent, and unlike the more differentiated epithelia elsewhere in the reproductive tract, retain the capacity to differentiate along several pathways. Nulliparity and hyperovulation treatment for infertility are epidemiologically associated with an increased risk of ovarian cancer, while pregnancies and oral contraceptives are associated with a decreased risk. These associations led to proposal of the incessant ovulation hypothesis for disease etiology (Fathalla, M. F. 1971. Lancet 2:163), whereby repeated ruptures of the ovarian epithelial surface increase the odds for transformation. It is known that inclusion cysts within the ovary are preferred sites for neoplastic progression. The presence of these cysts in some women suggests entrapment of surface epithelium during ovulation. There have also been reports, however, that inclusion cysts are numerous with less frequent ovulation and most numerous with polycystic ovarian syndrome, a condition associated with high androgen levels.

It has been suggested that cyst formation occurs via inflammatory adhesions of surface epithelium in which the resultant epithelial folds then involute (Scully, R. E. 1995. J. Cell. Biochem. 25(S):208). Regardless of the mechanism considered, the formation of cysts, which are devoid of the tunica albuginea barrier normally found at the ovarian surface, allow for the cystic epithelium to be influenced by the stromal environment, which includes hormone production, cells and soluble or matrix-derived factors.

Cytokines and chemokines may recruit cytokine-producing lymphocytes, including T-cells, to the developing follicle, and play an important role in follicle development (Nash, M. A. et al. 1999. Endocrine-Related Cancer 6:93). T-cells also reside within normal epithelia and are known to be important for the proliferation of epithelial cells. Thus, leukocytes and factors that influence leukocytes may be part of the initial environment necessary for development of ovarian tumors. In addition, cytotoxic T-cells, CD8+ cells, have been shown to be critical for effective anti-tumor responses. CD8+ cells are present throughout the female reproductive tract, including the human ovary.

In order for an ovarian tumor to be established, the CD8+ cells, or cytotoxic T-cells must not establish an effective anti-tumor response. Reasons for why T-cells fail to establish such anti-tumor responses may include immunologic ignorance, lack of access of immune cells to the tumor, and an insufficiently strong immune response or transient immune response due, for example, to ineffective priming or cross-priming of the antigen presenting complex (Ochsenbein, A. F. et al. 2001. Nature 411:1058). In the case of ovarian carcinoma, clinical pathology data showing that large numbers of leukocytes are seen to infiltrate the tumor site and the associated ascites fluid indicate that ovarian cancer patients mount a substantial, albeit ineffective, immune response to the tumor, an effect that allows for tumor establishment.

Researchers have investigated methods for stimulating effective anti-tumor cytotoxic T-cell responses (Melichar, B. and R. S. Freedman. 2002. Gynecolog. Cancer 12:3). Methods examined have included antigen stimulation using antigens expressed on ovarian carcinoma cells such as HER-2, MUC1, folate binding protein, and p53 (Peoples, G. E. et al. 1999. Clin. Can. Res. 5:4214), as well as administration of cytokines known to stimulate cytotoxic T cells such as interferon gamma and interleukin-2 or anti-estrogens (Freedman, R. S. et al. 2000. Clin. Can. Res. 6:2268; Baral, E. et al. 2000. Antican. Res. 20:2027). Although most of the research has focused on strategies for stimulation of a response rather than delineating the underlying mechanisms, Woo et al. (2001) demonstrated that increased percentages of ovarian tumor lymphocytes were CD4+ CD25+that could secrete TGFβ, a mechanism consistent with the role of T-cells in immune dysfunction in tumor tissues (Woo, E. Y. et al. 2001. Can. Res. 61:4766).

Antalarmin is a corticotropin releasing hormone (CRH) antagonist that has been used to treat a variety of conditions that involve either inflammatory responses or are stress-induced (Walk, M. et al. 2002. Gastroenterology 123:505-515; Gabry, K. E. et al. 2002. Mole. Psychiat. 7:474-483; Miller, D. B. and J. P. O'Callaghan. 2002. Metabolism 51:5-10; Webster, E. L. et al. 2002. J. Rheumatol. 29:1252-1261; Briscoe, R. J. et al. 2000. Brain Res. 881:204-207; Habib, K. E. et al. 2000. Proc. Natl. Acad. Sci. USA 97:6079-6084). The pharmacology of the compound has been linked to its ability to antagonize activity of CRH at its receptors. Recent studies, however, have shown that antalarmin administration decreased the number of implantation sites an live embryos in rodents (Mahrigiannakis, A. et al. 2001. Nature Immunol. 2:1018-1024). Antalarmin was shown to decrease fetal cell Fas ligand expression, resulting in decreased apoptosis of fetal cell-recognizing T-cells. Further, the data showed that CRH was important in maintaining pregnancy at the maternal-fetal interface by enabling fetal trophoblast cells to kill maternal cytotoxic T-cells that would presumably otherwise induce fetal rejection from uterine tissue.

It has now been found that otherwise ineffective T-cells derived from the ovarian tumor site can be rescued with respect to their T-cell lytic function through the use of antalarmin, thereby providing a new treatment for ovarian cancer, as well as other cancers where an anti-tumor response is effective.

### SUMMARY OF THE INVENTION

An object of the present invention is a composition for increasing cytotoxic T-cell lytic activity which comprises antalarmin and a pharmaceutically acceptable vehicle.

Another object of the present invention is a composition for treatment of human ovarian cancer which comprises antalarmin and a pharmaceutically acceptable vehicle.

Yet another object of the present invention is a method for increasing cytotoxic T-cell lytic activity which comprises measuring a first level of cytotoxic T-cell lytic activity in cells or tissues; contacting said cells or tissues with an effective amount of antalarmin and a pharmaceutically acceptable vehicle; and measuring a second level of cytotoxic T-cell lytic activity in said cells or tissues, wherein contacting of cells or tissues with antalarmin results in an increase in the second level of lytic activity as compared to the first level of lytic activity.

Another object of the present invention is a method for killing tumor cells in a tumor-bearing animal which comprises administering to a tumor-bearing animal an effective amount of antalarmin and a pharmaceutically acceptable vehicle wherein antalarmin increases cytotoxic T-cell lytic activity at tumor sites so that tumor cells are killed.

Another object of the present invention is a method of treatment of ovarian cancer comprising administering to a patient with ovarian cancer an effective amount of antalarmin and a pharmaceutically acceptable vehicle so that cytotoxic T-cell lytic or anti-tumor activity at ovarian tumor sites of the patient is increased and tumor cells are killed.

### DETAILED DESCRIPTION OF THE INVENTION

The present invention includes compositions and methods for treatment of ovarian carcinoma, as well as other types of tumors that rely on the ability of the tissue surrounding the tumor to stimulate an effective anti-tumor response involving cytotoxic T-cells. The compositions of the present invention comprise stress hormone modulating compounds and include the CRH antagonist, antalarmin. Antalarmin has now been shown to be an effective stimulator of immune cell rescue in tissue samples, a response that leads to induction of an anti-tumor response by the rescued immune cells that is otherwise diminished by the tumor cells present in the tissue or at the tumor site.

In the context of the present invention, “rescue” of a cell is defined as the act of restoring a function to a cell that is normally found associated with that cell in the non-tumor state. In the present invention the function to be restored is cytotoxic or cytolytic activity of T-cells or other anti-tumor activity by T-cells. Also in the context of the present invention, an “effective anti-tumor response” is defined as the ability of an agent or drug, such as antalarmin, to induce anti-tumor activity in cells within a tissue, where an effective response is one where there is a measurable decrease in the size of a tumor or in the growth rate of a tumor. A measurable decrease is one that can be shown experimentally in either cells in culture or in animals in vivo.

Studies were performed in cells from human pre-menopausal uterine endometrial tissue, non-tumor tissue, in order to first characterize the activity of T-cells in human tissues. It had been demonstrated that CD3+ T-cells are present in human pre-menopausal uterine endometrium, comprising about 50% of the leukocyte population, which in turn represented about 15 to 20% of the dispersed cells (Givan, A. L. et al. 1997. Am. J. Reprod. Immunol. 38:350). CD14+antigen presenting cells, likely accessory cells to T-cells, were also present throughout the reproductive tract (approximately 10 to 20% of the leukocytes). CD8+ T-cells outnumbered CD4+ cells in the benign reproductive tract tissues, in contrast to what is seen in peripheral blood. Freshly isolated lytic cells were tested for lytic function by an anti-CD3 monoclonal antibody (OKT3)-dependent redirected lysis assay. In this chromium release assay, the amount of chromium released by radiolabeled target cells into the supernatant fraction was measured to obtain the percent specific lysis or cytotoxicity (% specific lysis) mediated by cytotoxic T-cells. Lytic activity by cytotoxic T-cells was found throughout the reproductive tract, but was absent in the pre-menopausal uterine endometrial samples. In the post-menopausal samples and in the lower reproductive tract samples (vagina and cervix), CD8+ cells were strongly lytic. Thus, CD8+ activity was inversely correlated with ovarian hormone levels (estradiol and progesterone). The absence of lytic activity in the pre-menopausal samples was seen despite the presence of substantial numbers of CD8+ cells. These data are consistent with the premise that non-lytic CD8+ T-cells are required for reproduction to allow the semi-foreign fetus to avoid being rejected by maternal immune cells. The T-cells observed in the pre-menopausal samples can be termed “anergic” with respect to their low or non-existent cytolytic ability and are also referred to as non-responsive cells.

To determine whether the non-lytic pre-menopausal uterine endometrial T-cells were capable of lytic function, the overnight culture phase was lengthened to stimulate freshly isolated cells (whole populations of cells containing stromal cells, epithelial cells and leukocytes) for 3 days with plate bound anti-CD3 monoclonal antibody (to stimulate the T-cell receptor CD3 complex) and soluble anti-CD28 monoclonal antibody (to effect co-stimulation), followed by a 3 day resting phase, together referred to as “CD3 rescue” culture conditions. Lytic activity was seen in response to CD3 rescue at effector to target (E:T) ratios as low as 1:1. Redirected lysis assay samples were evaluated in parallel by FACS analysis to determine the CD3+ and CD8+ effector cell:target cell ratios and generally it was found that CD45+ leukocytes made up 15 to 20% of the cells, CD3+ cells made up 50% of the leukocytes, while CDB+:CD8− T-cell ratios were about 1.5:1. Using FACS analysis and cell dye uptake to monitor proliferation it was seen that T-cell lytic function was rescued and that lytic activity was not due to proliferation of a small, previously undetectable, lytic population. The levels of lysis seen under CD3+ rescue conditions were similar to the maximum levels obtained under non-inhibitory conditions, indicating that CD3+ rescue appeared to have fully restored cytotoxic T-cell lytic activity.

The ability of the cells to maintain the low or non-lytic state after separating T-cells from other cells was examined. To test for the presence of CD8− inhibitory cells, human pre-menopausal endometrial T-cells were separated into CD8+ and CD8− fractions using CD8− specific micromagnetic bead columns. Although not 100% pure, this fractionation system allowed for demonstration of functional differences between cell fractions. Unfractionated, CD8+ or CD8− cell fractions were cultured for 3 days in the absence or the presence of interleukin-2 (IL-2). Effector cell populations were then tested by redirected lysis assay and FACS analysis to allow determination of CD3+ versus CD8+ effector to target ratios. Results showed that IL-2 responsiveness was recovered in the CD8+ fraction. Thus, the non-lytic state of the T-cells was not due to the presence of CD8+ T-suppressor cells and the non-lytic or anergic state could be reversed.

The T-cell lytic function in human ovarian carcinoma cells was then tested in tissue from solid tumors and ascites, to allow comparison with the results seen in the benign tissues. This was done in order to determine if there were parallels in the requirement for non-lytic T-cells in the pre-menopausal endometrium (to avoid rejection of implanted embryos) and the tumor cells (to avoid tumor cell rejection).

Results showed that a large population of CD3+ T-cells was present in these tissues as a subset of CD45+leukocytes, is while there was a lower CD8+:CD8− T-cell ratio than was observed in the benign tissues. In addition, studies showed that cytotoxic T-cell activity was absent in the human ovarian carcinoma samples but that activity could be rescued using plate bound anti-CD3 monoclonal antibody and soluble phase anti-CD28 monoclonal antibody, as was shown for the benign, pre-menopausal tissue samples. Results with tumor samples versus ascites samples were similar. These data indicated that T-cells could be stimulated and that inhibitory pathways could be overcome. Further studies showed that, similar to the results obtained with the benign tissues, separation of CD8+ cells from CD8− cells led to restoration of IL-2 responsiveness, indicating that the non-lytic phenotype was due to inhibition by associated cells.

Experiments were then performed to examine the potential role of cytokines and co-stimulation in the non-lytic state of T-cells in human benign reproductive tract and ovarian tumor-associated tissues. The cytokines focused on were those known to affect CD8+ T cells and included TGFβ, interleukin-10 (IL-10), and interferon gamma. Samples of either benign endometrium or human ovarian carcinoma were cultured in the presence of IL-2 and other cytokines or anti-cytokine blocking antibodies for lengths of time varying from overnight to 6 days. In other experiments, T cells were stimulated with IL-2 with or without CD3 rescue conditions, as described above. Culture supernatant fractions were then tested for cytokine by ELISA, or cells were tested for cytokine by FACS analysis. Samples were tested either after the stimulation phase or at the end of the culture period. In parallel, cells were tested for lytic activity by CD3-dependent redirected lysis assay.

Results showed that the presence of blocking antibody to TGFβ in addition to IL-2 during culture of freshly isolated cells for 3 to 6 days partially restored T-cell lytic function in samples from benign pre-menopausal endometrial tissue. This rescue was partial with respect to the level of rescue is compared with CD3-stimulated rescue and with respect to the percent of samples rescued. These data indicated that TGFβ played at least some role in CD8+ lytic function. Blocking antibody to IL-10 consistently failed to restore lytic function in the pre-menopausal endometrial samples. These data were contrary to work of other investigators which had implicated IL-10 in stimulation of cytolytic activity in human cervical cancer samples (Santin, A. D. et al. 2000. J. Virol. 74:4729). Interferon gamma expression, as determined by ELISA, was shown to be increased by concomitant rescue of lytic function by culture of reproductive tract benign or ovarian tumor samples with plate bound anti-CD3 monoclonal antibody (CD3 rescue). In addition, the presence of few interferon gamma positive T-cells but many IL-10 positive T-cells in ascites fluid was consistent with the idea that lack of interferon gamma production is related to the dysfunctional T-cells response in ovarian carcinoma.

Considering the combined data, it was clear that factors other than cytokines were involved in control of lytic activity in cytotoxic T-cells in human reproductive tract tissues, including human ovarian carcinoma cells. Experiments were then performed to examine the role of potential endocrine factors in regulating the responsiveness of T-cells.

CRH is known to be an important endocrinologic factor that is produced by tumor cells, and/or by associated leukocytes, in response to tumor cells. Therefore, experiments were performed to investigate the role of CRH pathways in maintaining the lytic state of T-cells and the potential for agents that modulate this pathway to affect T-cell lytic function. To facilitate the experimentation process, a mouse model system was employed that is a model for human ovarian cancer disease (Roby, K. F. et al. 2000. Carcinogenesis 21:585). In this model, mouse ovarian surface epithelial cells were obtained from mature, virgin mice and repeatedly passaged in vitro, about 20 passages, until cobblestone morphology and contact inhibition of growth were lost. Clonal tumor lines were established from these experiments. Injection of these tumor cells intraperitoneally into syngeneic mice (C57BL6) resulted in formation of tumor nodules and ascitic fluid, as well as progression of disease similar to that seen in humans. For example, the location of tumors, how they sit within the peritoneum and how they spread is similar between mouse and human. Further, like human ovarian tumors, the mouse tumors are estrogen receptor alpha positive, src tyrosine kinase is constitutively upregulated, c-fms-1 gene is intact and expressed, and they are positive for urokinase plasminogen activator.

The first experiments performed were designed to characterize the T-cell lytic state of the murine ovarian carcinoma cells. Using techniques described above for human cells, the populations of T-cells were examined from ascites-derived cell samples. It was seen that leukocytes were approximately 50% of all ascites cells and that there was a substantial population of CD3+ T-cells in the ascites samples (20 to 30%). CD3+ CD8− cells outnumbered CD3+ CD8+cells. CD3− leukocytes and tumor cells were also present. Thus, the population profiles were similar to those in human ovarian carcinoma samples, samples with T-cells not in a lytic state.

To assess the IL-2 responsiveness of T-cells within murine ascites, whole populations of cells were cultured overnight for up to 3 days with IL-2 and then tested for lytic activity using the redirected lysis assay. The assay was similar to the assay described for human cells but an anti-mouse CD3 monoclonal antibody (2C11) was used instead of OKT3. While control splenic cells from non-tumor bearing mice contained cytotoxic T-cell lytic function, the ascites samples had no IL-2 responsive T-cell lytic activity. To determine whether there was activity in murine ascites that was inhibitory to T-cell lytic function, as opposed to simply cells that had been rendered inactive, washed ascites cells were co-cultured with spleen cells from a non-tumor bearing mouse for 3 days in the presence of IL-2. Only background levels of lytic activity were observed, indicating that the murine ascites-derived cells inhibited splenic cytotoxic T-cells lytic function. An attempt to stimulate splenic T-cells via CD3 rescue in the presence of ascites-derived cells resulted in partial rescue (20% lysis). These data showed that murine ascites samples contained a source of inhibitory cells.

Experiments were then performed with antalarmin, a CRH receptor antagonist, to determine if manipulation of CRH activity was involved in the regulation of lytic activity in ovarian carcinoma cells. Spleen cells from normal non-tumor bearing mice were co-cultured in vitro for 3 days with tumor ascites inhibitory cells from the mouse model system. The cells were then tested for immunologic function of cytotoxic T-cells using the redirected lysis assay. Upon stimulation of splenic cytotoxic T-cells (with anti-CD3/CD28 monoclonal antibodies), tumor ascites inhibitory cells down-regulated cytotoxic T-cell lytic function, while antalarmin (0.5 to 1 micromolar) prevented the down-regulation. These data support that mechanism whereby tumor cells produce CRH, which in turn upregulates a killing receptor that kills cytotoxic T-cells, a process that is blocked by antalarmin. Antalarmin was also shown to prevent the loss of CD3− stimulated T-cells that was mediated by murine ascites inhibitory cells; stimulated splenic T-cells (CD3+ and CD8+) were lost when co-cultured with tumor ascites inhibitory cells, an effect that was not seen in the presence of antalarmin (0.5 to 1 micromolar).

Experiments also showed that antalarmin prevented the loss of spleen cells at early stages of ovarian carcinoma disease. Normal C57BL6 mice were challenged with ID8 murine ovarian carcinoma cells (intraperitoneally) in the presence of control vehicle (diluent), antalarmin, T-cell stimulation with 2C11 antibody, or antalarmin plus T-cell stimulation. Ten days after challenge, spleen cells were harvested and T-cells were restimulated with either antibody to the T-cell receptor and co-receptor or ID8 tumor cells in the presence of antalarmin, antibody stimulation, or a combination of antalarmin plus antibodies to T-cell receptor and co-receptor. Six days later, cells were harvested and tested for splenic cell yields. The addition of antalarmin to the tumor cells for the in vivo priming phase followed by non-specific stimulation or no stimulation in the restimulation phase, resulted in the best yields of spleen cells. Treatment with tumor cells plus antalarmin plus antibody stimulation in the in vivo priming phase, followed by restimulation with irradiated tumor cells in the presence of antalarmin, provided conditions also favorable for preventing spleen cell loss induced by tumor cells. Thus, antalarmin was an important factor in preserving immune cells in vivo in the initial phases of ovarian cancer disease.

The present invention, therefore, is a composition for induction or increasing of cytotoxic T-cell lytic activity and killing of tumor cells, as well as a treatment for ovarian cancer in humans. It is through the increase in the cytotoxic T-cell lytic activity that tumor cells are killed and thus cancer is treated. The composition comprises a stress hormone modulating compound. Any compound that has the ability to modulate, in particular antagonize, the effects of stress hormones is contemplated by the present invention to have potential as a treatment for ovarian cancer. In a preferred embodiment this stress hormone modulating compound is a CRH antagonist, antalarmin, that is combined with a pharmaceutically acceptable vehicle for delivery of the drug. One of skill would choose such pharmaceutically acceptable vehicles based upon knowledge generally available in the art. A generally recognized compendium of methods and ingredients of pharmaceutically acceptable vehicles is Remington: The Science and Practice of Pharmacy, Alfonso R. Gennaro, editor, 20th ed. Lippingcott Williams & Wilkins: Philadelphia, Pa., 2000.

The present invention is also a method for inducing or increasing cytotoxic T-cell lytic activity or anti-tumor activity and for killing of tumor cells comprising administration of an effective amount of antalarmin so that lytic activity is increased in populations of cytotoxic T-cells, resulting in tumor cell death. In this method, the effect of antalarmin in cells is measured by first determining a baseline level of cytotoxic T cell activity in cells or tissues (referred to as a first level of cytotoxic activity) and then measuring cytotoxic T cell activity after rescue of the cells by antalarmin (referred to as the second level of activity). Methods for determining levels of cytotoxic T cell activity are provided herein and are well-known to those of skill in the art.

Treatment of a patient having ovarian cancer with antalarmin is by administration of an effective amount of antalarmin and a pharmaceutically acceptable vehicle. An effective amount of antalarmin is considered an amount that increases cytotoxic T-cell lytic activity or anti-tumor activity at the ovarian tumor site of said patient and said tumor cells are killed, wherein the term “patient” includes humans. Increases in cytotoxic T-cell lytic activity may be determined as described herein and anti-tumor activity and killing of tumor cells may be assessed using well-established methods such as assessing tumor size, feelings of weakness, and pain perception. It is contemplated that antalarmin and pharmaceutically acceptable vehicle may be administered orally, for example in the form of pills, tablets, lacquered tablets, coated tablets, granules, hard and soft gelatin capsules, solutions, syrups, emulsions, suspensions or aerosol mixtures. Administration may also be carried out parenterally (e.g., intravenously, intramuscularly, subcutaneously in the form of injection solutions or infusion solutions, microcapsules, implants or rods); or percutaneously or topically (e.g., in the form of ointments, solutions, emulsions or tinctures, patches, bandages or liquid bandages). A composition of the invention may also be administered into body orifices such as the rectum and vagina in the form of a suppository or cream.

The selected pharmaceutically acceptable vehicle may be dependent on the route of administration and may be an inert inorganic and/or organic carrier substance and/or additive. For the production of pills, tablets, coated tablets and hard gelatin capsules, the pharmaceutically acceptable carrier may include lactose, corn starch or derivatives thereof, talc, stearic acid or its salts, and the like. Pharmaceutically acceptable vehicles for soft gelatin capsules and suppositories include, for example, fats, waxes, semisolid and liquid polyols, natural or hardened oils, and the like. Suitable carriers for the production of solutions, emulsions, or syrups include, but are not limited to, water, alcohols, glycerol, polyols, sucrose, glucose, and vegetable oils. Suitable carriers for microcapsules, implants or rods include copolymers of glycolic acid and lactic acid.

A composition of the invention, in general, contains about 0.5 to 90% by weight of antalarmin. The amount of antalarmin in the composition normally is from about 0.1 mg to about 1000 mg, preferably from about 1 mg to about 500 mg.

In addition to antalarmin and a pharmaceutically acceptable vehicle, the composition of the invention may contain an additive or auxiliary substance. Exemplary additives include, for example, fillers, disintegrants, binders, lubricants, wetting agents, stabilizers, emulsifiers, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

Those of ordinary skill in the art may readily optimize effective doses and co-administration regimens as determined by good medical practice and the clinical condition of the individual patient. Regardless of the manner of administration, it may be appreciated that the actual preferred amounts of active compound in a specific case will vary according to the particular formulation and the route of administration. The specific dose for a particular patient depends on age, body weight, general state of health, on diet, on the timing and route of administration, on the rate of excretion, and on medicaments used in combination and the severity of the particular disorder to which the therapy is applied. Dosages for a given subject may be determined using conventional considerations, e.g., by customary comparison of the differential activities of the subject compound and of a known agent, such as by means of an appropriate conventional pharmacological protocol. Generally, an amount between 0.1 mg/kg and 100 mg/kg body weight/day of antalarmin.

### EXAMPLES

### Example 1

### Redirected Lysis Assay

Freshly isolated reproductive tract cells were tested for lytic function by an anti-CD3 mAb (OKT3)-dependent redirected lysis assay (White et al., J. Immunology 158:3017, 1997). In this chromium release assay, anti-CD3 mAb vs control mAb (3-5 μg/mL) was bound by its Fc end to Fc Receptor-bearing ⁵¹Cr-labeled P815 target cells (murine mastocytoma cell line) for 30 minutes at 37° C., after which the mAb-bound P815 target cells were incubated for 6 hours with CD3+ effector cell-containing populations from the reproductive tract. The chromium released into the supernatant fraction was measured to obtain the percent specific cytotoxicity (% specific lysis), i.e. percent of labeled target cells killed by CD3+ effector cells, using duplicate wells and a titration of 3 effector to target ratios. The percent specific lysis was calculated by standard methods, briefly as follows: % specific lysis=(experimental cpm−spontaneous release cpm)/(freeze-thaw releasable total cpm), where experimental cpm is the ⁵¹Cr released from target cells in the presence of effector cells, spontaneous release cpm is the ⁵¹Cr released from target cells in the absence of effector cells, and total cpm is the total ⁵¹Cr releasable from target cells (determined to be −80% of the total counts per minute by cycles of freezing and thawing). Previous experiments proved the specificity of CD3− specific CTL activity using the isotype control Ab W6/32 which binds to a monomorphic determinant of MHC Class I and thus conjugates T-cells to P815 target cells through an antigen other than the TCR but fails to result in lytic activity by endometrial effector cells.

### Example 2

### Rescue of T-Cell Function

CD3 rescue of cytotoxic T-cell function. Reproductive tract T-cells were cultured for 3-4 days with plate bound anti-CD3 mAb (10 μg/1 mL was bound to each well for 1 hour 37° C. or overnight 4° C., then excess Ab washed off 3× with 3-5 mL media prior to plating cells) in the presence of 10 U/mL IL-2 and 0.3 μg/mL anti-CD28 mAb (initial stimulation), rested in culture for 3-4 days without any stimulus (“rest phase” after washing cells and replating into fresh wells with culture medium), and finally cultured a second time in culture medium with 10 U/mL IL-2 (without anti-CD3 Ab) overnight (restimulation phase) prior to the 6 hour chromium release assay. As a control, IL-2 without anti-CD3 mAb was present for the initial stimulation.

An alternative rescue of cytotoxic T-cell function by purification away from inhibitory cells. Reproductive tract-derived cells were separated into CD8+ and CD8− negative fractions using Miltenyi CD8 specific micromagnetic bead columns (Miltenyi Biotec, Inc, Auburn, Calif.), according to the manufacturer's protocol. Basically, Ab bound micromagnetic beads were bound to cells for 15 minutes at 10° C., excess beads washed off, and bead-bound cells passed over a magnetic column mounted next to a magnet. CD8− negative cells passed through; the column-retained CD8+ cells were obtained by taking the column away from the magnet and eluting the CD8+ cells with column buffer. CD8+ cells were then cultured for 3 days in 10 U/mL IL-2 containing culture medium in the presence or absence of CD8-negative inhibitory cells at a ratio of approximately 10:1 CD8+ cell: inhibitory cell prior to assessment by redirected lysis assay to obtain the IL-2 responsive lytic function phenotype.

### Example 3

### Cytokine Assays

Reproductive tract-derived cells were stimulated either by CD3 rescue (above), or using standard methods to stimulate cytokine production: 20 ng/mL PMA/1 μM ionomycin 4-6 hours to test for IL-2 or IFNgamma production; and 1 μg/mL LPS 24 hours to test for IL-10 production.

Cytokine secretion was tested by standard Enzyme-linked Immunoassay (ELISA) paired capture and detection Ab kits (R&D Systems, Minneapolis, Minn.) in which 2-4 μg/mL capture Ab (100 μL well) is used to bind cytokine, and 100-500 ng/mL enzyme linked detection Ab is used to quantitate the cytokine of interest relative to a standard curve with known concentration of cytokine in accordance with standard protocols.

Intracellular cytokine expression was assessed by FACS (fluorescence activated cell sorter) analysis using standard staining and detection methods. 10 μg/mL Brefeldin A was added 4 hours prior to the end of the stimulation protocol to promote retention of cytokine within cells prior to FACS analysis. For FACS analysis, cell non-specific sites were blocked with Ig, cells stained for surface antigen, e.g. 1 μg anti-CD3 or anti-CD8 antibody per 10⁶ cells for 30 minutes at 4° C., then washed and treated with 0.1% saponin to allow entry of Ig block and then 1 μg anti-cytokine Ab (30 minutes, 4° C.) to the intracellular compartment; cells were then washed and fixed in 1% paraformaldehyde, and assessed by FACS analysis.

### Example 4

### Mouse Model System

Tumor-associated ascites was induced in vivo by injection i.p. of a C57BL6-derived ovarian carcinoma cell line ID8 (Roby et al, Carcinogenesis 21:585, 2000) into C57BL6 female mice and harvesting ascites samples approximately 4 weeks after injection. Initial experiments used the whole population of cells from ascites samples, which contained CD8+ T cells, CD3+ T regulatory-like cells and macrophages, in addition to other leukocytes as well as tumor cells. CD3+ CD8+T cells within ascites samples were anergic, i.e. they failed to have lytic activity in the redirected lysis assay using P815 target cells provided to them (redirected lysis assay, above, but with anti-murine CD3 mAb to conjugate effector cells to FcR bearing target cells and thereby assess the ability of ascites-derived CD8+ T cells to kill). Ascites cells were demonstrated to have strong inhibitory activity when co-cultured with normal C57BL6 splenic cells for 3 days (assessment by redirected lysis assay); for these experiments, cells were co-cultured in medium containing 10 U/ml IL-2 with or without plate-bound anti-CD3 mAb and soluble anti-CD28 Ab (CD3 rescue, above) prior to lytic assay or FACS analysis. FACS analysis of splenic CD3+ cells and CD8+ cells (demonstrating that ascites-derived inhibitory cells caused the loss of normal splenic T cells), used directly labeled mAbs to CD3 and CD8 and standard multicolor fluorometric staining methods.

What is claimed is:

1. A composition for increasing cytotoxic T-cell lytic activity comprising antalarmin and a pharmaceutically acceptable vehicle.

2. A composition for treatment of human ovarian cancer comprising antalarmin and a pharmaceutically acceptable vehicle.

3. A method for increasing cytotoxic T-cell lytic or anti-tumor activity comprising:
a) measuring a first level of cytotoxic T-cell lytic activity in cells or tissues;
b) contacting said cells or tissues with an effective amount of antalarmin and a pharmaceutically acceptable vehicle; and
c) measuring a second level of cytotoxic T-cell lytic activity in said cells or tissues, wherein contacting of cells or tissues with antalarmin results in an increase in the second level of lytic activity as compared to the first level of lytic activity.

4. The method of claim 3 wherein said cells or tissues are human ovarian carcinoma cells.

5. A method for killing tumor cells in a tumor-bearing animal comprising administering to a tumor-bearing animal an effective amount of antalarmin and a pharmaceutically acceptable vehicle wherein antalarmin increases cytotoxic T-cell lytic activity or anti-tumor activity at tumor sites so that said tumor cells are killed.

6. The method of claim 5 wherein said tumor cells are ovarian carcinoma cells.

7. A method of treatment of ovarian cancer comprising administering to a patient with ovarian cancer an effective amount of antalarmin and a pharmaceutically acceptable vehicle so that cytotoxic T-cell lytic activity or anti-tumor activity at the ovarian tumor site of said patient is increased and said tumor cells are killed.
